(19) United States
(12) Patent Application Publication
(10) Pub. No.: US 2002/0031481 A1
(43) Pub. Date: Mar. 14, 2002

(54) Stable herbal dentrifice

(76) Inventors: Jin Xu, Livingston, NJ (US); Bala C. Nayar, Dayton, NJ (US); Marissa Healy, Denville, NJ (US)

Correspondence Address: Nora Stein-Fernandez, Esq., SmithKline Beecham corporation, 709 Swedeland Road, UW2220, P.O. Box 1539, King of Prussia, PA, 19406-0939, US

(21) Appl. No.: 09/838,021

(22) Filed: Apr. 19, 2001

Related U.S. Application Data
(63) Continuation-in-part of application No. 09/419,515, filed on Oct. 18, 1999, now abandoned.
Application No. 09/419,515 is a continuation-in-part of application No. 09/134,462, filed on Aug. 14, 1998, now abandoned.
Application No. 09/134,462 is a continuation-in-part of application No. 09/031,951, filed on Feb. 27, 1998, now abandoned.

(51) Int. Cl. A61K 7/16

(52) U.S. Cl. 424/49

(57) ABSTRACT

A dentifrice gel or paste comprising from about 40% to 70% by weight baking soda, from about 0.5% to 5% by weight of a blend of natural ingredients, from about 0.5% to 5% by weight of a flavoring oil and a sufficient amount of a stabilizing combination of a xanthan gum binder, at least one betaine surfactant and a humectant comprising glycerin or glycerin and sorbitol in an amount sufficient to render the dentifrice formulation stable to degradation and resistant to syneresis and phase separation.

### RELATED APPLICATIONS

This application is a continuation-in-part of U.S. patent application Ser. No. 09/419,515, filed Oct. 18, 1999, which is a continuation-in-part of U.S. patent application Ser. No. 09/134,462, filed Aug. 14, 1998, which is a continuation-in-part of U.S. patent application Ser. No. 09/031,951, filed Feb. 27, 1998.

This invention relates to oral health care products, particularly dentifrices, containing fluoride, baking soda and optionally one vitamin or vitamin precursor, that are stable to degradation and resistant to syneresis and phase separation.

### BACKGROUND OF THE INVENTION

Dentifrices have long been known and used to clean teeth and to combat plaque. Formulators constantly seek new ways to improve the attractiveness of a dentifrice to encourage compliance with an oral health care regimen. In addition to flavor improvements, various visual improvements have been made over the traditional white pastes. Such an improvement is the dentifrice gels which provided strong appeal to the public and encouraged compliance with daily oral health care. Another was the increasing use of “natural” ingredients, including baking soda as well as other natural or herbal ingredients.

One problem with using baking soda as a dentifrice ingredient, however, is stability, especially at higher levels. Baking soda (sodium bicarbonate) is soluble in water and has a tendency to break down when associated with water at higher temperatures thus reducing shelf life to an unacceptably short time. This effect may be accelerated by the presence of other ingredients in dentifrice formulations. Other natural or herbal ingredients that provide desirable benefits for dentifrices may also suffer from stability problems as well.

Numerous means of stabilizing baking soda in a dentrifice have been proposed. In U.S. Pat. Nos. 3,937,804 and 3,943,240, about 20%, preferably 30%, baking soda is mixed with water, a polyol humectant, a gelling or thickening agent and fluoride in a dentifrice that has a granular textured appearance. Stability appears to be maintained because the baking soda is separated from the water content by forming large granules. In contrast, U.S. Pat. Nos. 4,623,536 and 4,721,614 are directed to a toothpaste containing at least 60% sodium bicarbonate, at least 30% of the which has a particle size of less than 25 microns. The smaller particle size is asserted to be the basis for the claimed stability of the dentifrice. U.S. Pat. No. 4,943,429 discloses a dentifrice gel containing up to about 60% by weight sodium bicarbonate and at least about 22% of a humectant such as glycerol or sorbitol.

Sodium bicarbonate and peroxide dentifrices have been provided in split-tube designs to avoid stability problems, as in U.S. Pat. No. 5,456,902. U.S. Pat. Nos. 5,180,576 and 5,318,773 disclose dentifrices incorporating baking soda and pyrophosphate salts (tartar control agents), which is asserted to have synergistic properties, permitting the use of reduced amounts of pyrophosphate salts. The two agents must be isolated in the product because the baking soda “salts out” the pyrophosphate, leaving little pyrophosphate to dissolve in the dentifrice before use.

In regard to the inclusion of other natural ingredients in dentrifice compositions, U.S. Pat. No. 5,472,684 discloses a combination providing plaque and gingivitis treatments comprising thymol, eugenol and optionally a sesquiterpene alcohol, such as famesol, as an antibacterial agent. The combination is flavored with various natural agents such as chamomile, myrrh gum, rhatany root, Australian tea tree oil, and the like. The solid portion of the dentifrice is a gelling agent such as carboxymethyl cellulose, xanthan gum, sodium alginate, methyl cellulose, and the like. U.S. Pat. No. 4,812,306 discloses a water-free toothpaste containing anise oil, clove oil, sassafras, peppermint, glycerin, corn starch, fluoride, sweetener, sodium lauryl sulfate, sodium bicarbonate, and vegetable oil.

A major problem inherent in the inclusion of natural ingredients in dentrifice preparations is syneresis, i.e. the tendency of the liquid components to separate from solids. Syneresis can be especially acute in formulations containing high solid levels, such as are found in dentifrices containing baking soda. The problem becomes even more acute when the dentrifice preparation also contains natural ingredients in liquid form, such as various tinctures and extracts, flavor oils such as peppermint oil, and the like. For example. a leading commercial dentrifice which comprises about 67% sodium bicarbonate, less than 2% each of myrrh, rhatany, chamomile, eichinacea, sage oil, mint oil, sodium lauryl sulfate, about 1000 ppm sodium fluoride with a pH level of about 8.3 is prone to syneresis which renders it undesirable.

In addition to the agents described above, especially unstable materials that have been proposed in dentifrices and mouthwashes are the vitamins. U.S. Pat. No. 5,188,817, for example, teaches the use of Vitamin A in tooth paste or tooth gel compositions for treating periodontitis. U.S. Pat. No. 5,009,886 discloses the use of vitamins (C, A, and D) as flavor enhancers in dentifrices, with the preferred composition comprising sodium lauryl sulfate as a surfactant.

Vitamin E is susceptible to degradation in most aqueous vehicles, and a number of approaches have been tried to minimize its degradation in oral health care products. U.S. Pat. No. 4,292,304, for example, uses a substantially anhydrous, oil-based dentifrice composition that includes a source of Vitamin E, and an abrasive agent. This material is stored in an edible capsule. Anhydrous dentifrices, however, can have an unpleasant mouthfeel and a reduced shelf life due to oxidation of an oil-based solvent. U.S. Pat. No. 4,411,885 attempts to solve these problems by making a dentifrice in a tablet, which is actually a compact mass of a substantially anhydrous composition containing at least 10% by weight Vitamin E.

Vitamin E is often used in its precursor acetate ester form that is hydrolyzed in vivo to release the vitamin. In addition, Vitamin E salts have been utilized in cosmetic compositions (U.S. Pat. No. 5,683,704); as an antibacterial component, a vitamin component and a surfactant component in a toothpaste, mouthwash or chewing gum (U.S. Pat. No. 3,992,519). Japanese Patent Abstract Publication No. 63-192712 to Shinji et al. discloses a toothpaste containing Vitamin E wherein stability is improved though the use of a container having an inner surface of a synthetic resin.

U.S. Pat. No. 5,747,005 discloses an oil-based anti-plaque dentifrice containing Vitamin E and an enzyme, with the use of thickening agents as such as polyethylene glycol and sodium lauryl sulfate. It is stated therein (col. 4, lines 61-67) that gum compositions are not sufficiently compatible with the Vitamin E and enzyme components to be adequately resistant to dilution and wash-away in the presence of saliva.

Contrary to the prior art teaching, the inventors have found a particular gum thickening agent in combination with a particular surfactant and a humectant unexpectedly renders dentifrice compositions containing a high level of baking soda and other natural ingredients stable to degradation and that the stability is sustained even with the added presence of oil-based vitamin components.

### SUMMARY OF THE INVENTION

The invention provides a dentifrice composition comprising 40 to 70% by weight sodium bicarbonate, from about 0.5 to 5% by weight of a blend of natural or herbal ingredients, from about 0.5 to 5% by weight of a flavoring oil, and a sufficient amount of a stabilizing combination of from about 0.3 to 3.0% by weight of a xanthan gum binder, from about 0.5 to 5.0% by weight of at least one betaine surfactant, preferably cocamidopropyl betaine, and from about 5 to 20% by weight of at least one humectant, preferably glycerol, to render the dentifrice formulation stable to degradation and resistant to syneresis.

The invention further provides a method of stabilizing a dentifrice formulation containing a high level of sodium bicarbonate, and a blend of natural ingredients against degradation and resistant to syneresis by blending therein a stabilizing combination of xanthan gum, at least one betaine surfactant and at least one humectant.

### DESCRIPTION OF THE PREFERRED EMBODIMENTS

To overcome the degradation and syneresis problem described above, the invention provides a dentifrice comprising a high level of baking soda, a blend of natural or herbal ingredients, optionally at least a vitamin or vitamin precursor, flavors and a stabilizing combination of three ingredients blended to form a stable dentifrice.

The dentifrice of the invention comprises a very high level, i.e. from about 40 to 70% by weight baking soda (sodium bicarbonate). Wherein the dentifrices of the invention are translucent gels, they contain from about 40 to about 50% by weight of baking soda. Most preferably, baking soda comprises from about 45 to 50% by weight of the dentifrice for an aesthetically pleasing translucent gel. It should be noted that the problem of syneresis and degradation encountered with the Parodontax® formula was not as severe when it was formulated as a gel because, in order to obtain the gel appearance, it was necessary to reduce the baking soda content. However, it still occurred in sufficient frequency to be considered a problem in the commercial environment.

Wherein the dentifrices produced in accordance with the present invention are in the form of an opaque toothpaste, they contain from about 60 to about 70% by weight of baking soda. Preferably, such formulations contain from about 65 by weight to about 70% by weight of baking soda, thereby producing an aesthetically pleasing opaque paste. Depending on the amount of baking soda and the particle size classification thereof based on sieve classification, the dentifrice may be in a paste or gel form. A preferred gel formulation contains baking soda with not more than about 2% by weight retained on a No. 100 (150 μm) standard screen, about 20-45% retained on a No. 200 (75 μm) standard screen, and about 60-100% retained on a No. 325 (45 μm) standard screen. This particle size distribution is especially useful in producing an aesthetically pleasing gel dentifrice.

A preferred opaque dentifrice paste contains baking soda with a finer particle size distribution, e.g., not more than about 2% by weight retained on a No. 100 (150 μm) standard screen, not more than about 5% by weight retained on a No. 200 (75 μm) standard screen, about 17-27% by weight retained on a No. 325 (45 μm) standard screen and the remainder passing therethrough.

In addition to baking soda, the dentifrice comprises various natural ingredients. The term “natural ingredient” as used herein means substances extracted or derived, typically, but not exclusively, from plants with minimal chemical alteration or processing. Such substances include plant extracts, pressings from herbs and flowers, distillations of various materials, and the like. These are known substances, many of which are commercially available.

A preferred combination of natural ingredients is that in the commercial dentifrice Parodontax® which comprises echinacea, myrrh, chamomile, rhatany and sage. Other natural or herbal ingredients include, without intended limitation, aloe, houttuynia, glycyrrhiza and gardenia extracts.

Echinacea, also known as cone flower juice or extract, is described as the dried rhizome and roots of Echinacea pallida, and comprises insulin, sucrose, betaine and other ingredients. Myrrh, which is derived from various species of Commiphora, is a combination of a gum, a volatile oil and a bitter principle. Chamomile, prepared from the dried flower heads of Anthemis nobilis, comprises a bitter glucoside, anthesterol, anthemene and a volatile oil. Rhatany, also known as Krameria, is the dried root of Krameria triandra and other Krameria plants. It comprises Krameriatannic acid. Sage, also known as Salvia, is a widely known flavoring agent. It comprises 1-2.5% volatile oil, resin, tannin, and bitter principles.

Aloe is a material derived from the leaves of the aloe plant, e.g., Aloe barbadansis Miller, Aloe argorescens Miller, Aloe africana Miller or Aloe spicata Baker. Aloe contains cathartic anthraglycosides which help promote wound healing. Houttuynia, derived from Hyuttuynia cordata, contains aldehydes and ketones. It is known and used as a natural anti-inflammatory agent. Glycyrrhiza, a known flavoring agent, is derived from the roots of Glycyrrhiza uralensis and contains glycyrrhizin. Gardenia extract comes from the fruit of Gardenia jasminoides Ellis (Rubiaceae). The extract is used in perfume, food coloring and as an antipyretic. Most of these natural substances are commercially available, primarily in the form of a tincture, which is an alcoholic solution of nonvolatile materials.

Although dentifrice preparations in accordance with the present invention can contain a specific natural ingredient, a blend of a number of natural ingredients as described above is preferred. In a preferred embodiment of the invention, the natural ingredient blend is a combination of the Parodontax® brand dentifrice natural ingredients as listed above, although other natural ingredients may be included as well.

In a preferred embodiment, the natural ingredients may comprise from about 0.5 to about 5% by weight of the dentifrice. In a more preferred embodiment, the natural ingredients may comprise from about 1 to about 4% by weight of the dentifrice. A particularly preferred formulation contains about 3.5% by weight a combination of natural ingredients including those present in Parodontax® brand dentifrice.

The dentifrice preparations of the present invention contain a suitable flavor. These are typically natural oils such as spearmint, peppermint and the like, with peppermint oil being preferred. The formulations of the invention are advantageous in that they may contain a large amount of one or more flavoring oils, i.e. from about 0.5 to 5%, preferably from about 2 to 3% by weight of the subject formulations.

It has been found in accordance with the present invention that dentifrice preparations containing a high percentage of baking soda and a blend of natural ingredients can be stabilized against degradation and phase separation by the inclusion of a specific stabilizing combination of xanthan gum, a betaine surfactant and one or more humectants. The fact that formulations such as the Parodontax® brand dentifrice formulation can be stabilized by the specific combination of the invention is considered unexpected in view of the fact that the marketed formulation prior to the modification thereof to include the subject combination of stabilizing ingredients contained ingredients generically similar to those making up the subject combination, yet was characterized by significant degradation and syneresis.

Specific examples of the betaine surfactant utilized in the combinations of the invention include cocamidopropyl betaine, ricinoleamidopropyl betaine, stearyl betaine, lauric myristic betaine, cocoamidosulfobetaine, alkylamidophospho betaine and alkyldimethylbetaines in which the alkyl group contains from 8 to 18 carbon atoms. The preferred betaine is cocamidopropyl betaine. Typical humectants include glycerin, sorbitol, propylene glycol and polyethylene glycol. The preferred humectant is glycerin alone or in combination with another humectant, preferably sorbitol.

The dentifrice preparations according to the invention may comprise from about 5 to about 20%, preferably from about 5 to 10%, by weight of the humectant, from about 0.3 to 3% by weight, preferably about 1.5% by weight of the xanthan gum and from about 0.5 to 5%, preferably about 4% by weight of the betaine surfactant.

In addition to the foregoing essential ingredients, the dentifrice preparations of the present invention preferably contain other ingredients typically present in such formulations including, without intended limitation, a source of fluoride ion, one or more vitamins or vitamin precursors, emulsifying agents, sweeteners, polishing agents, abrasives, whitening agents, anti-bacterials, anti-tartar agents and desensitizing compounds, coloring agents and the like. Examples of suitable sources of fluoride ion include sodium fluoride, stannous fluoride and sodium monofluorophosphate. The fluoride source typically comprises less than about 2%, preferably from about 0.1 to 1% by weight of the dentifrice. Examples of suitable emulsifiers, both natural and synthetic, include phospholipids and lysophosphatidyl compounds such as lysolecithin, lysophosphatidyl-ethanolamine, lysophosphatidylcholine, lysophosphatidylglycerol, and the like. A preferred emulsifier is a naturally derived lysolecithin that can comprise up to about 2% by weight of the dentifrice.

Vitamins that may be incorporated into the subject formulations include any known vitamin, e.g. Vitamin A, the B vitamins, Vitamin C, Vitamin E and others, including precursors thereof that yield the vitamins in vivo. Examples of such precursors include esters, salts and other forms of vitamins that will yield usable forms of the vitamin upon hydrolysis or other chemical alteration in the body. A preferred vitamin is Vitamin E (also known as alpha-tocopherol) in the acetate or succinate form, preferably the former. The most highly preferred precursor is Vitamin E acetate. The vitamin or precursor may comprise up to about 3% by weight, preferably up to about 2% by weight, and most preferably up to about 1% by weight of the dentifrice formulation.

While Vitamin E is particularly desirable for inclusion in dentifrice preparations, because it is an oil, its presence increases the tendency for the preparation to separate and undergo syneresis, particularly where a dentifrice already contains a mixture of natural ingredients and one or more flavoring oils as described above. Therefore, in order to maintain a dentifrice stable against degradation and syneresis, it is necessary to reduce the concentration of sodium bicarbonate in the subject preparations to about 50% by weight or less in order to accommodate an oily vitamin, such as Vitamin E.

Examples of suitable polishing agents and abrasives include, without intended limitation, precipitated amorphous silica, various silicates such as magnesium silicate, calcium carbonate, di-calcium phosphate, alumina and the like. Such compounds may be used alone or in combination, and generally comprise from about 6 to about 12% by weight, preferably about 10% by weight of the dentifrice. Suitable sweeteners include, for example, sodium saccharin, sucrose, lactose, maltose, stevioside and the like. Sweetening agents generally comprises less than about 1% by weight of the subject dentifrice formulations.

The dentifrice of the invention can be prepared in accordance with techniques known to those of ordinary skill in the art of pharmaceutical compounding as illustrated in the following examples that are not in any intended to be limiting on the scope of the appended claims.

### EXAMPLE 1

A dentifrice was prepared having the ingredients set forth in Table 1. A 7,000 gram batch of toothpaste was prepared with Koruma® toothpaste making equipment. The top of the Koruma® was charged with purified water, sodium saccharin and sodium fluoride, and mixing continued for two minutes (Disho at 2,000 rpm). An 80 gram portion of the sodium bicarbonate was added and mixed for five minutes (Disho at 2,000 rpm). Glycerin and xanthan gum were then added with high shear and mixing continued for 10 minutes (Disho at 3,000) under vacuum, maintaining temperature at about 25° C. to 30° C. The myrrh tincture, chamomile extract, and coneflower tincture were added and mixing continued for three minutes (Disho at 3,000 rpm). Additional sodium bicarbonate, 2,200 g, was then added in four portions with mixing for two minutes after each addition (Disho at 2,500 rpm and vacuum at 600 mbar). A further 1,200 grams of sodium bicarbonate, premixed with the iron oxide pigment, was then added in two portions with two minutes of mixing after each addition (Disho at 2,100 rpm and vacuum at 600 mbar). Peppermint oil and sage oil were then added and mixed for one minute (Disho at 2,000 rpm). A further 390 grams of sodium bicarbonate were added and mixed for two minutes (Disho at 1,700 rpm and vacuum at 600 mbar). The rhatany tincture was added and mixed for one minute (Disho at 1,700 rpm). The remaining sodium bicarbonate (810 grams) was then added in two portions with mixing for two minutes after each addition (Disho at 1,700 rpm and vacuum at 600 mbar). The lid was opened and the walls of the Koruma were scraped. Mixing continued for ten minutes under full vacuum and Disho at 1,500 rpm. The batch was then placed into a suitable container for filling.

The dentrifice prepared as above is designated Formula A in Table 1. Four additional formulations that differed by the addition of ingredients as shown in Table 1 were also prepared. Two samples of each formulation were stored in coextruded tubes and the stability of the dentifrice was measured at 75% relative humidity and at 40° C. after four months. The toothpastes were found to be surprisingly chemically and physically stable (very little, if any, separation of the ingredients).

TABLE 1
Formulations of Example 1 in Weight Percent

| | Formula | Formula | Formula | Formula | Formula |
|---|---|---|---|---|---|
| Ingredient | A | B | C | D | E |
| Baking soda | 67.262 | 67.262 | 67.262 | 67.262 | 67.262 |
| Xanthan gum | 0.85 | 0.8 | 0.75 | 0.75 | 0.75 |
| Cocamidopropyl Betaine, | 4 | 4 | 4 | 4 | 4 |
| 30% | | | | | |
| Iron oxide E172 | 0.003 | 0.003 | 0.003 | 0.003 | 0.003 |
| Sodium fluoride | .310 | .310 | .310 | .310 | .310 |
| Sodium saccharin | 0.0164 | 0.0164 | 0.0164 | 0.0164 | 0.0164 |
| Glycerin | 5.540 | 5.540 | 5.540 | 5.540 | 5.540 |
| Coneflower tincture | 0.954 | 0.954 | 0.954 | 0.954 | 0.954 |
| Myrrh tincture | 0.624 | 0.624 | 0.624 | 0.624 | 0.624 |
| Chamomile tincture | 0.624 | 0.624 | 0.624 | 0.624 | 0.624 |
| Rhatany tincture | 1.248 | 1.248 | 1.248 | 1.248 | 1.248 |
| Peppermint oil | 1.926 | 1.926 | 1.926 | 1.926 | 1.926 |
| Sage oil | 0.146 | 0.146 | 0.146 | 0.146 | 0.146 |
| Zinc citrate trihydrate | — | 2. | — | — | — |
| Silica | — | — | 5 | — | — |
| Thymol/Eucalyptol/ | — | — | — | 0.4 | — |
| Eugenol | | | | | |
| Licorice extract | — | — | — | — | 0.4 |
| Purified water | Balance | Balance | Balance | Balance | Balance |

A portion of the Formula A above was placed into a 25 ml toothpaste tube and subjected to a standard stability testing. The results are reported in Table 2. The notation “NR” in Table 2 means no reading was taken. “Conforms” means that the tested property fell within the design specification for the product including no significant or observable physical separation of the ingredients. For appearance and container integrity the criteria was no obvious defect.

TABLE 2
Stability Tests on the Dentifrice of Example 1 (Formula A)

| Property Tested | Initial | 1 Month | 3 Months | 4.5 Months |
|---|---|---|---|---|
| Appearance | Conforms | Conforms | Conforms | Conforms |
| pH | 8.1 | 8.0 | 8.2 | 8.5 |
| Taste | Control | NR | Conforms | Conforms |
| Viscosity (× 10⁵), cps | 2.44 | 2.48 | 3.46 | 3.46 |
| Ionic Fluoride (ppm) | 1400 | 1296 | 1410 | 1489 |
| Total Volatile Oils, | 1.7 | 1.6 | 1.6 | 1.4 |
| v/w % | | | | |
| Sodium Bicarbonate, | 68.1 | 68.8 | 69.1 | 70.5 |
| w/w % | | | | |
| Primary Container | Conforms | Conforms | Conforms | Conforms |
| Integrity | | | | |
| Separation | None | None | None | None |
| Extrudability | Easy | Easy | Easy | Easy |

### EXAMPLE 2

For comparison against the stabilized formulations of the present invention, the results of a series of stability tests preformed on the original Parodontax® product are given in Table 3. It will be evident from the results in Table 3 that the Parodontax® formulation demonstrated separation almost immediately and exhibited further signs of degradation at three months.

TABLE 3
Stability Tests on Parodontax ® Marketed Formula

| Property Tested | Initial | 1 Month | 3 Months | 4.5 Months |
|---|---|---|---|---|
| Appearance | Conforms | Conforms | Color | Color faded |
| | | | faded | |
| pH | Conforms | Conforms | Conforms | Conforms |
| Taste | Control | N R | Conforms | Conforms |
| Viscosity | Conforms | Conforms | Conforms | Conforms |
| Ionic Fluoride (ppm) | Conforms | Conforms | Conforms | Conforms |
| Total Volatile Oils | Conforms | Conforms | Conforms | Conforms |
| Sodium Bicarbonate | Conforms | Conforms | Conforms | Conforms |
| Primary Container | Conforms | Conforms | Conforms | Conforms |
| Integrity | | | | |
| Separation | Severe | Severe | Severe | Severe |
| Extrudability | Easy | Easy | Moderate | Moderate |

For comparison purposes, the formula of Formula A of Example 1 and the original Parodontax® formulation are shown in Table 4.

TABLE 4
Formulations in Weight Percent

| Ingredient | Formula A | Parodontax ® |
|---|---|---|
| Baking soda | 67.262 | 67.262 |
| Xanthan gum | 0.850 | — |
| Carboxymethylcellulose Sodium | — | 1.200 |
| Cocamidopropyl Betaine, 30% | 4.000 | — |
| Iron oxide E172 | 0.003 | — |
| Sodium fluoride | .310 | .310 |
| Sodium saccharin | 0.0164 | 0.0164 |
| Glycerin | 5.540 | 5.540 |
| Coneflower tincture | 0.954 | 0.954 |
| Myrrh tincture | 0.624 | 0.624 |
| Chamomile tincture | 0.624 | 0.624 |
| Rhatany tincture | 1.248 | 1.248 |
| Peppermint oil | 1.926 | 1.926 |
| Sage oil | 0.146 | 0.146 |
| Sodium Lauryl Sulfate | — | 1.642 |
| Medicinal Soap | — | 0.256 |
| Erythrosine E127 | — | 0.0009 |
| Purified water | Balance | Balance |

What is claimed is:

1. An oral health care formulation comprising:
from about 40 to 70 wt. of sodium bicarbonate;
from about 0.5 to 5 wt. % of a blend of natural ingredients;
from about 0.5 to 5 wt. % of a flavoring oil; and
a sufficient amount of a stabilizing combination of a xanthan gum binder, at least one betaine surfactant and at least one humectant consisting of glycerin or glycerin and sorbitol to render the formulation stable to degradation and resistant to syneresis.

2. An oral health care formulation in accordance with claim 1, wherein said humectant is glycerin.

3. An oral health care formulation in accordance with claim 1, wherein said at least one betaine surfactant is selected from the group consisting of: ricinoleamidopropyl betaine, cocamidopropyl betaine, stearyl betaine, lauric myristic betaine, cocoamidosulfobetaine, alkylamidophospho betaine, and alkyldimethylbetaines in which the alkyl group contains 8-18 carbon atoms.

4. An oral health care formulation in accordance with claim 3, wherein said at least one betaine surfactant is selected cocamidopropyl betaine.

5. An oral health care formulation in accordance with claim 1, wherein said stabilizing combination comprises, based on the weight of said formulation, from about 0.5 to 5% of said xanthan gum binder, from about 0.3 to 3% of said at least one betaine surfactant, and from about 5 to about 20% of said humectant.

6. An oral health care formulation in accordance with claim 1, wherein said stabilizing combination comprises, based on the weight of said formulation, about 1.5% of said xanthan gum binder, about 4% of said at least one betaine surfactant, and from about 5 to about 10% of said humectant.

7. An oral health care formulation in accordance with claim 1, wherein said formulation is a dental gel containing from about 40 to about 50% by weight of sodium bicarbonate.

8. An oral health care formulation in accordance with claim 1, wherein said formulation is a dental paste containing from about 60 to about 70% by weight of sodium bicarbonate.

9. An oral health care formulation in accordance with claim 1, further comprising a fluoride source.

10. An oral health care formulation in accordance with claim 9, wherein said fluoride source is present in from about 0.1 to 1.0% by weight and is selected from the group consisting of sodium fluoride, stannous fluoride and sodium monofluorophosphate.

11. An oral health care formulation in accordance with claim 1, wherein said flavoring oil is peppermint oil.

12. An oral health care formulation in accordance with claim 1, wherein said blend of natural ingredients comprises from about 1 to 4% by weight of said formulation and comprises echinacea, myrrh, chamomile, rhatany and sage.

13. An oral health care formulation in accordance with claim 12, wherein said blend of natural ingredients comprises about 3.5% by weight of said formulation.

14. An oral health care formulation comprising:
from about 40 to 70% by weight sodium bicarbonate;
from about 0.5 to 5% by weight of a blend of natural ingredients comprising echinacea, myrrh, chamomile, rhatany and sage;
from about 0.5 to 5% by weight of a flavoring oil comprising peppermint oil;
a stabilizing combination to render said formulation stable against degradation and resistant to syneresis comprising, based on the weight of said formulation:
from about 0.5 to 5% by weight of xanthan gum binder;
from about 0.3 to 3% by weight of at least one betaine surfactant; and
from about 5 to about 20% by weight of at least one humectant consisting of glycerin or glycerin and sorbitol.

15. A method or rendering an oral health formulation stable against degradation and resistant to syneresis, wherein said composition comprises: from about 40 to 70% by weight of sodium bicarbonate; from about 0.5 to 5% by weight of a blend of natural ingredients; from about 0.5 to 5% by weight of a flavoring oil; and at least a surfactant, a binder and a humectant, said method comprising formulating said formulation including a specific combination of xanthan gum as the binder, at least one betaine as the surfactant and glycerin or glycerin and sorbitol as the humectant.
